(19) United States
(12) Patent Application Publication
(10) Pub. No.: US 2007/0048797 A1
(43) Pub. Date: Mar. 1, 2007

(54) Composite organic inorganic nanoclusters as carriers and identifiers of tester molecules

(76) Inventor: Xing Su, Cupertino, CA (US)

Correspondence Address: INTEL/BLAKELY, 12400 WILSHIRE BOULEVARD, SEVENTH FLOOR, LOS ANGELES, CA, 90025-1030, US

(21) Appl. No.: 11/527,895

(22) Filed: Sep. 26, 2006

Related U.S. Application Data
(63) Continuation-in-part of application No. 11/081,772, filed on Mar. 15, 2005.
Application No. 11/081,772 is a continuation-in-part of application No. 10/940,698, filed on Sep. 13, 2004.
Application No. 10/940,698 is a continuation-in-part of application No. 10/916,710, filed on Aug. 11, 2004.

(51) Int. Cl. G01N 33/53; C12Q 1/37

(52) U.S. Cl. 435/7.1; 977/902; 435/23

(57) ABSTRACT

Metallic nanoclusters capable of providing an enhanced Raman signal from an organic Raman-active molecule incorporated therein are provided. The nanoclusters are generally referred to as COINs (composite organic inorganic nanoparticles) and are capable of acting as sensitive reporters for analyte detection. Embodiments of the invention provide methods for detecting and quantitating enzyme activity. Further, the parallel assay capabilities of COINs allow libraries of compounds and molecules to be tested for enzyme activity.

### CROSS REFERENCE TO RELATED APPLICATIONS

The present invention is a continuation-in-part of U.S. patent application Ser. No. 11/081,772, filed Mar. 15, 2005, which is a continuation-in-part of U.S. patent application Ser. No. 10/940,698, filed Sep. 13, 2004, now pending, which is a continuation-in-part of U.S. patent application Ser. No. 10/916,710, filed Aug. 11, 2004, now pending, and U.S. patent application Ser. No. 11/021,682, filed Dec. 23, 2004, now pending, which are continuation-in-parts of U.S. patent application Ser. No. 10/830,422, filed Apr. 21, 2004, now pending, which is a continuation-in-part of U.S. patent application Ser. No. 10/748,336, filed Dec. 29, 2003, now pending, and the disclosures of which are considered part of and are incorporated by reference in the disclosure of this application. The present application is also related to U.S. patent Applications No. 11/027,470, filed Mar. 2, 2006, now pending, No. 11/026,857, filed Dec. 30, 2004, now pending, No. 11/325,833, filed Dec. 30, 2005, now pending, and No. 11/216,112, filed Sep. 1, 2005, now pending, the disclosures of which are incorporated herein by reference

### BACKGROUND OF THE INVENTION

1. Field of the Invention

Embodiments of the invention relate generally to nanoclusters that include metal particles and organic compounds and to the use of such nanoclusters in analyte detection by surface-enhanced Raman spectroscopy.

2. Background Information

The ability to detect and identify trace quantities of analytes has become increasingly important in many scientific disciplines, ranging from part per billion analyses of pollutants in sub-surface water to analysis of drugs and metabolites in blood serum. Additionally, the ability to perform assays in multiplex fashion greatly enhances the rate at which information can be acquired. Devices and methods that accelerate the elucidation of disease origin, creation of predictive and or diagnostic assays, and development of effective therapeutic treatments are valuable scientific tools. A principle challenge is to develop an identification system for a large probe set that has distinguishable components for each individual probe.

Among the many analytical techniques that can be used for chemical analyses, surface-enhanced Raman spectroscopy (SERS) has proven to be a sensitive method. A Raman spectrum, similar to an infrared spectrum, consists of a wavelength distribution of bands corresponding to molecular vibrations specific to the sample being analyzed (the analyte). Raman spectroscopy probes vibrational modes of a molecule and the resulting spectrum, similar to an infrared spectrum, is fingerprint-like in nature. As compared to the fluorescent spectrum of a molecule which normally has a single peak exhibiting a half peak width of tens of nanometers to hundreds of nanometers, a Raman spectrum has multiple structure-related peaks with half peak widths as small as a few nanometers.

To obtain a Raman spectrum, typically a beam from a light source, such as a laser, is focused on the sample generating inelastically scattered radiation which is optically collected and directed into a wavelength-dispersive spectrometer. Although Raman scattering is a relatively low probability event, SERS can be used to enhance signal intensity in the resulting vibrational spectrum. Enhancement techniques make it possible to obtain a 10⁶ to 10¹⁴ fold Raman signal enhancement.

SERS effect is attributed mainly to electromagnetic field enhancement and chemical enhancement. It has been reported that silver particle sizes within the range of 50-100 nm are most effective for SERS. Theoretical and experimental studies also reveal that metal particle junctions are the sites for efficient SERS.

### BRIEF DESCRIPTION OF THE FIGURES

FIGS. 1A and B show Raman spectra obtained from COINs (composite organic inorganic nanoclusters) that incorporate a single type of Raman label and three different Raman labels, respectively. (Key: 8-aza-adenine (AA), 9-aminoacridine (AN), methylene blue (MB).) Representative peaks are indicated by arrows; peak intensities have been normalized to respective maximums; the Y axis values are in arbitrary units; spectra are offset by 1 unit from each other.

FIGS. 2A and B show signatures of COINs with double and triple Raman labels. The three Raman labels used were 8-aza-adenine (AA), 9-aminoacridine (AN), and methylene blue (MB). The main peak positions are indicated by arrows; the peak heights (in arbitrary units) were normalized to respective maximums; spectra are offset by 1 unit from each other.

FIGS. 3A, B, and C show Raman spectra from several organic Raman labels and COINs.

FIG. 4 is an enzymatic activity assay scheme in which COINs are used as carriers for an enzyme substrate.

FIG. 5 shows an in vitro method for screening drugs or other molecules for effectiveness as inhibitors or activators of enzymes.

FIG. 6 provides another method for screening molecules or compounds for biologic activity in which the molecule(s) and or compound(s) are attached to COINs.

FIG. 7 provides a further method for screening molecules or compounds for biologic activity in which the molecule(s) and or compound(s) are attached to COINs.

### DETAILED DESCRIPTION OF THE INVENTION

Generally, composite organic inorganic nanoclusters (COINS) are composed of a metal and at least one organic Raman-active compound. Interactions between the metal of the clusters and the Raman-active compound(s) enhance the Raman signal obtained from the Raman-active compound(s) when the nanoparticle is excited by a laser. COINs according to embodiments of the invention can function as sensitive reporters for use in analyte detection. Since a large variety of organic Raman-active compounds can be incorporated into the nanoclusters, a set of COINs can be created in which each member of the set has a Raman signature unique to the set. Thus, COINs can also function as sensitive reporters for highly parallel analyte detection. Furthermore, not only are the intrinsic enhanced Raman signatures of the nanoparticles of the present invention sensitive reporters, but sensitivity may also be further enhanced by incorporating thousands of Raman labels into a single nanocluster and or attaching multiple nanoclusters to a single analyte.

Aggregated metal colloids fuse at elevated temperature and organic Raman labels can be incorporated into the coalescing metal particles. These coalesced metal particles form stable clusters and produce intrinsically enhanced Raman scattering signals from the incorporated organic label(s). These stable clusters containing organic molecules incorporated within and as part of the cluster are COINs.

The interaction between the organic Raman label molecules and the metal colloids of the nanoparticle cluster has mutual benefits. Besides serving as signal sources, the organic molecules induce a metal particle association that is in favor of electromagnetic signal enhancement. Additionally, the internal nanocluster structure provides spaces to hold Raman label molecules, especially in the junctions between the metal particles that make up the cluster. In fact, it is believed that the strongest enhancement is achieved from the organic molecules located in the junctions between the metal particles of the nanoclusters.

Generally, the nanoclusters are less than 1 μm in size, and are formed by particle growth in the presence of organic compounds. The preparation of such nanoparticles also takes advantage of the ability of metals to adsorb organic compounds. Indeed, since Raman-active organic compounds are adsorbed onto the metal cluster during formation of the metallic colloids, many Raman-active organic compounds can be incorporated into a nanoparticle.

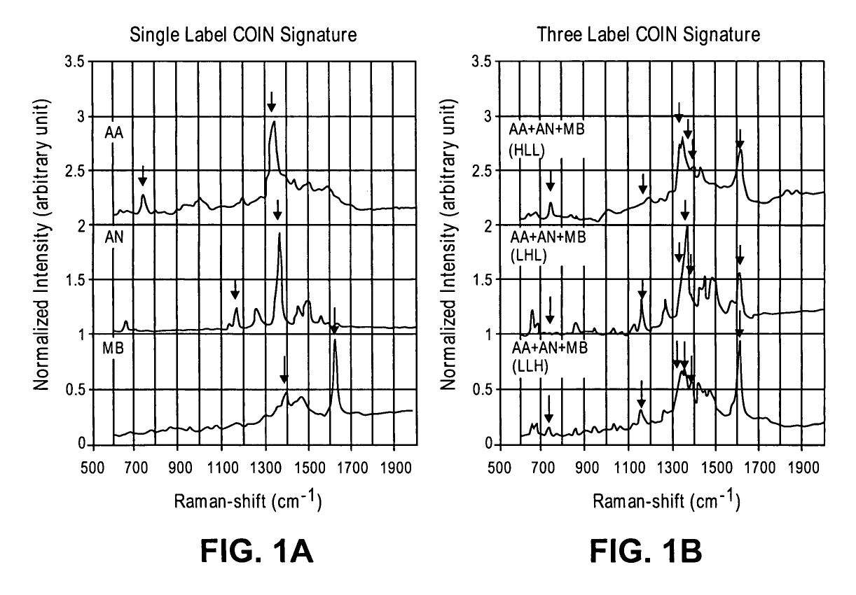

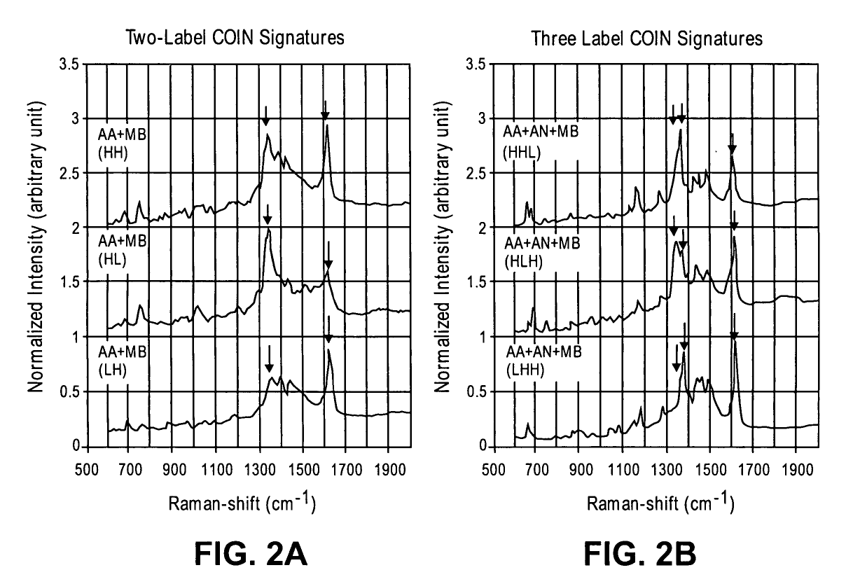

Not only can COINs be synthesized with different Raman labels, but COINs may also be created having different mixtures of Raman labels and also different ratios of Raman labels within the mixtures. Referring now to FIGS. 1 and 2, FIG. 1A shows signatures of COINs made with a single Raman-active organic compound, demonstrating that each Raman-active organic compound produced a unique signature. FIG. 1B shows signatures of COINs made with mixtures of three Raman labels at concentrations that produced signatures as indicated: HLL means high peak intensity for 8-aza-adenine (AA) (H) and low peak intensity for both 9-aminoacridine (AN) (L) and methylene blue (MB) (L); LHL means low peak intensity for AA (L), high peak intensity for AN (H) and low for MB (L); LLH means low for both AA (L) and AN (L) and high for MB (H). COINs in these examples were made with individual or mixtures of Raman labels at concentrations from 2.5 μM to 20 μM, depending on the signature desired. Peak heights can be adjusted by varying label concentrations, but they might not be proportional to the concentrations of the labels used due to different absorption affinities of the Raman labels for the metal surfaces. FIG. 2A shows signatures of COINs made with 2 Raman labels (AA and MB) at concentrations designed to achieve the following relative peak heights: AA=MB (HH), AA>MA (HL), and AA<MB (LH). FIG. 2B shows Raman signatures of COINs made from mixtures of the three Raman labels at concentrations that produced the following signatures: HHL means high peak intensities for AA (H) and AN (H) and low peak intensity for MB (L); HLH means high peak intensity for AA (H), low peak intensity for AN (L), and high peak intensity for MB (H); and LLH means low peak intensities for AA (L) and AN (L), and high peak intensity for MB (H). COINs in these examples were made by the oven incubation procedure with mixtures of two or three Raman labels at concentrations from 2.5 to 20 μM, depending on the signatures desired. Thus, it is possible to create a large number of different molecular identifiers using the COINs of the present invention. Theoretically, over a million COIN signatures could be made within the Raman shift range of 500-2000 cm⁻¹.

Table 1 provides examples of the types of organic compounds that can be used to build COINs. In general, Raman-active organic compound refers to an organic molecule that produces a unique SERS signature in response to excitation by a laser. Typically the Raman-active compound has a molecular weight less than about 500 Daltons.

TABLE 1

| No. | Abbreviation | Name | Structure |
|---|---|---|---|
| 1 | AAD (AA) | 8-Aza-adenine | 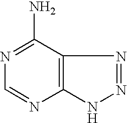 |
| 2 | BZA (BA) | N-Benzoyladenine | 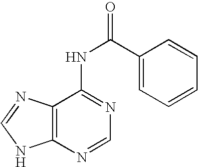 |
| 3 | MBI | 2-Mercapto-benzimidazole | 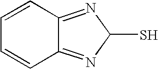 |
| 4 | APP | 4-Amino-pyrazolo[3,4-d]pyrimidine | 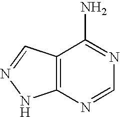 |
| 5 | ZEN | Zeatin | 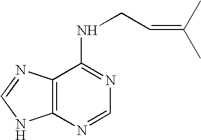 |
| 6 | MBL (MB) | Methylene Blue | 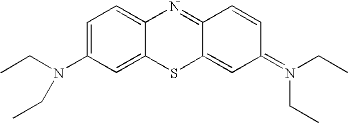 |
| 7 | AMA (AN, AM) | 9-Amino-acridine | 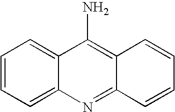 |
| 8 | EBR | Ethidium Bromide | 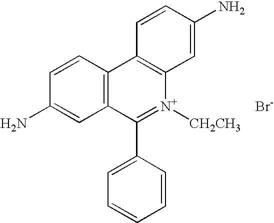 |
| 9 | BMB | Bismarck Brown Y | 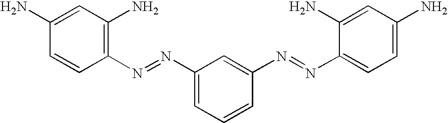 |
| 10 | NBA | N-Benzyl-aminopurine | 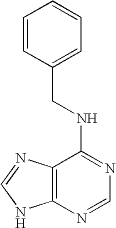 |
| 11 | THN | Thionin acetate | 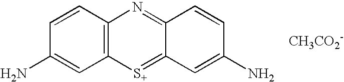 |
| 12 | DAH | 3,6-Diaminoacridine | 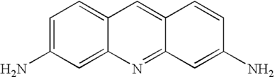 |
| 13 | CYP | 6-Cyanopurine | 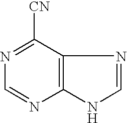 |
| 14 | AIC | 4-Amino-5-imidazole-carboxamide hydrochloride | 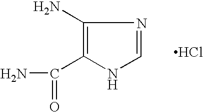 |
| 15 | DII | 1,3-Diiminoisoindoline | 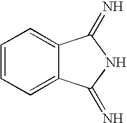 |
| 16 | R6G | Rhodamine 6G | 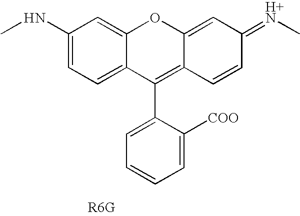 |
| 17 | CRV | Crystal Violet | 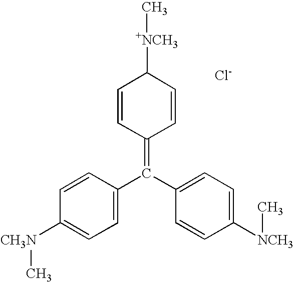 |
| 18 | BFU | Basic Fuchsin | 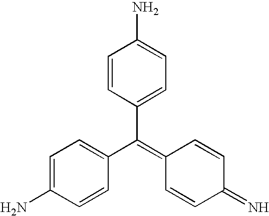 |
| 19 | ANB | Aniline Blue Diammonium salt | 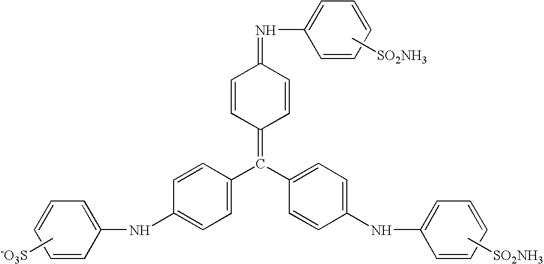 |
| 20 | ACA | N-[(3-(Anilinomethylene)-2-chloro- 1-cyclohexen-1- yl)methylene]aniline monohydrochloride | 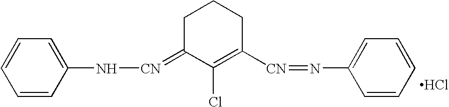 |
| 21 | ATT | O-(7-Azabenzotriazol-1-yl)- N,N,N′,N′-tetramethyluronium hexafluorophosphate | 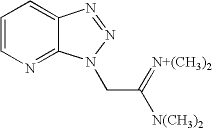 |
| 22 | AMF | 9-Aminofluorene hydrochloride | 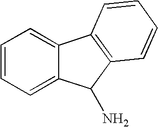 |
| 23 | BBL | Basic Blue | 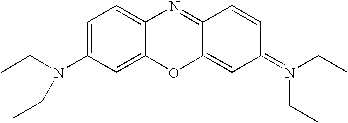 |
| 24 | DDA | 1,8-Diamino-4,5- dihydroxyanthraquinone | 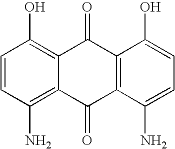 |
| 25 | PFV | Proflavine hemisulfate salt hydrate | 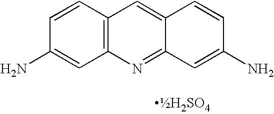 |
| 26 | APT | 2-Amino-1,1,3- propenetricarbonitrile | 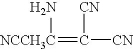 |
| 27 | VRA | Variamine Blue RT salt | 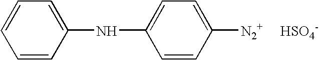 |
| 28 | TAP | 4,5,6-Triaminopyrimidine sulfate salt | 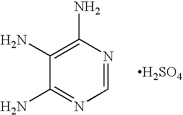 |
| 29 | ABZ | 2-Amino-benzothiazole | 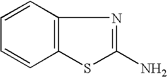 |
| 30 | MEL | Melamine | 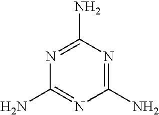 |
| 31 | PPN | 3-(3-Pyridylmethylamino) Propionitrile | 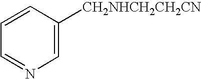 |
| 32 | SSD | Silver(I) Sulfadiazine | 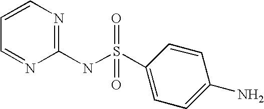 |
| 33 | AFL | Acriflavine | 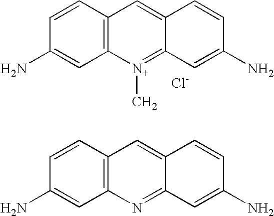 |
| 34 | AMPT | 4-Amino-6-mercaptopyrazolo[3,4- d]pyrimidine | 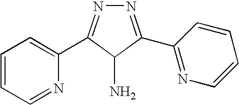 |
| 35 | APU | 2-Aminopurine | 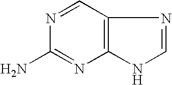 |
| 36 | ATH | Adenine Thiol | 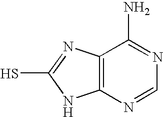 |
| 37 | FAD | Fluoroadenine | 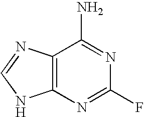 |
| 38 | MCP | 6-Mercaptopurine | 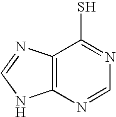 |
| 39 | AMP | 4-Amino-6-mercapyopyrazolo[3,4- d]pyrimidine | 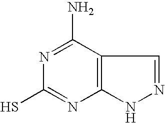 |
| 41 | R110 | Rhodamine 110 | 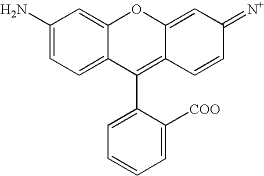 |
| 42 | ADN | Adenine | 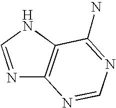 |
| 43 | AMB | 5-Amino-2-mercaptobenzimidazole | 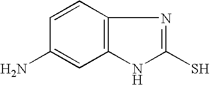 |

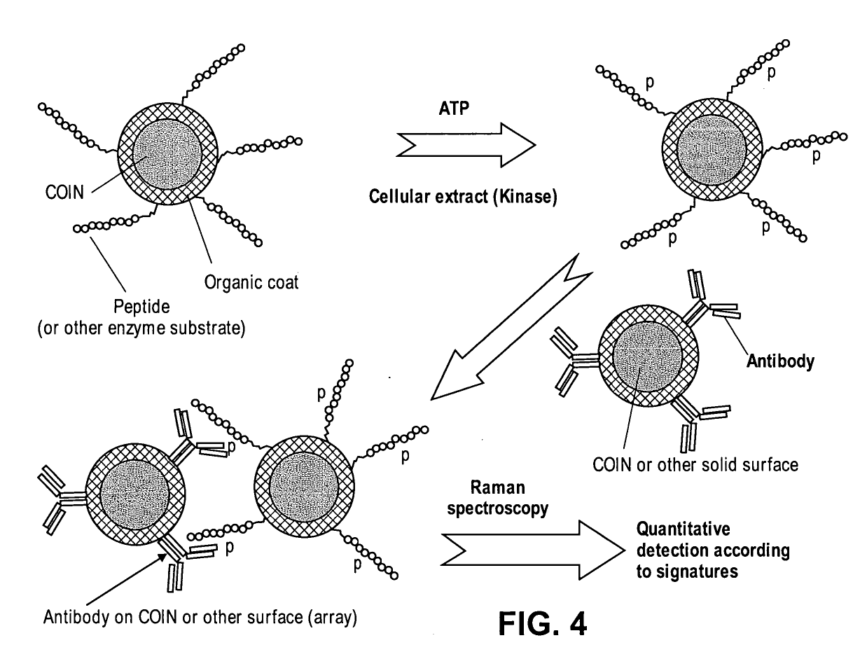

Many compounds that give strong regular Raman signals in solution do not yield strong signals in COINs. Further, within a particular compound, vibration modes that give strong regular Raman peaks in solution do not necessarily produce strong peaks in COINs. Strong signals from COINs are desirable in applications such as the detection of analytes that are present at low concentrations. It was found, for example, that the organic compounds shown in Table 2 produced strong Raman signals upon incorporation into the COIN nanocluster. Referring now to FIG. 4, a comparison is provided between signal intensities achieved for COINs containing several different organic compounds. The vertical axis plots the label type and the abbreviations can be found in Table 2.

TABLE 2

| No. | Abbreviation | Name | Structure |
|---|---|---|---|
| 44 | AOH | Acridine Orange Hydrochloride | 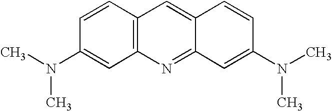 |
| 45 | CVA | Cresyl Violate Acetate | 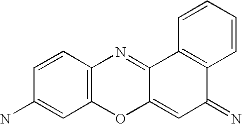 |
| 46 | AFN | Acriflavine Neutral | 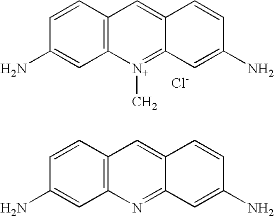 |
| 47 | DMB | Dimidium Bromide | 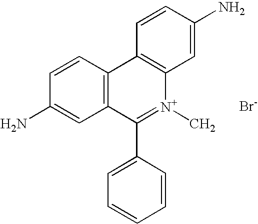 |
| 48 | TMP | 5,10,15,20-Tetrakis(N-methyl-4- pyridinio)porphyrin Tetra(p- toluenesulfonate) | 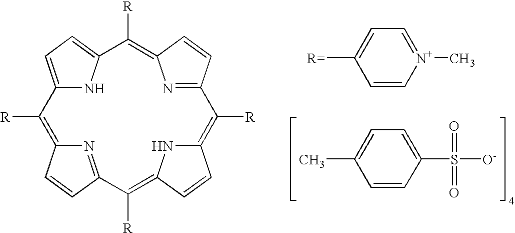 |
| 49 | TTP | 5,10,15,20-Tetrakis(4- trimethylaminophenyl)porphyrin Tetra(p-toluenesulfonate) | 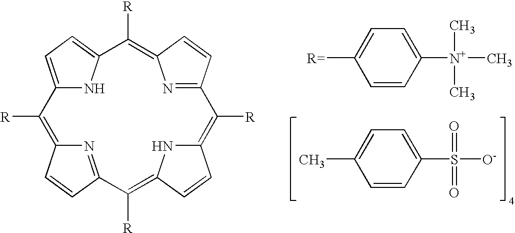 |
| 50 | DAA | 3,5-Diaminoacridine Hydrochloride | 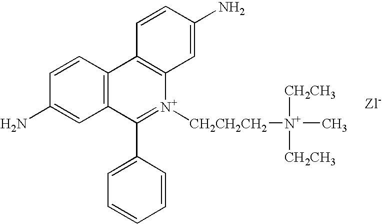 |
| 51 | PII | Propidium Iodide (3,8-diamino-5-(3- diethylaminopropyl)-6- phenylphenanthridinium iodide methiodide) | 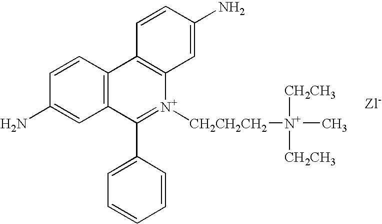 |
| 52 | MPI | Trans-4-[4-(dimethylamino)styryl]-1- methylpyridinium iodide | 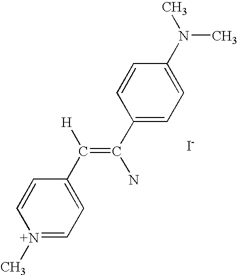 |
| 53 | DAB | 4-((4- (dimehtylamino)phenyl)azo)benzoic acid, succinimidyl ester | 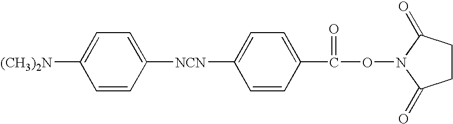 |

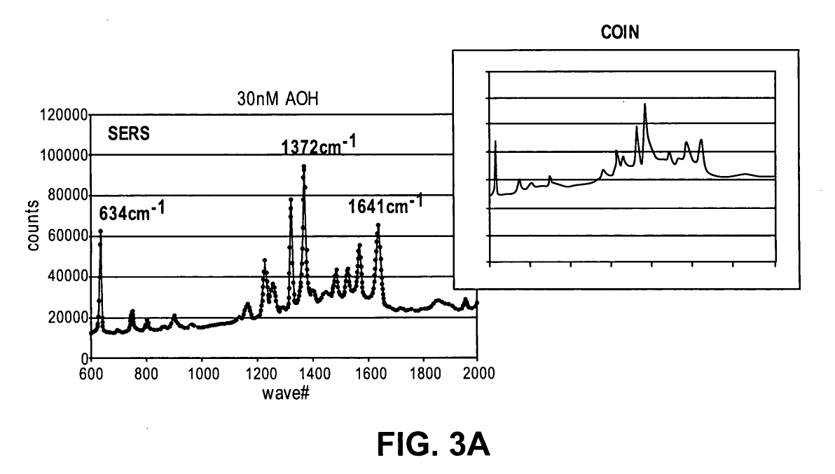

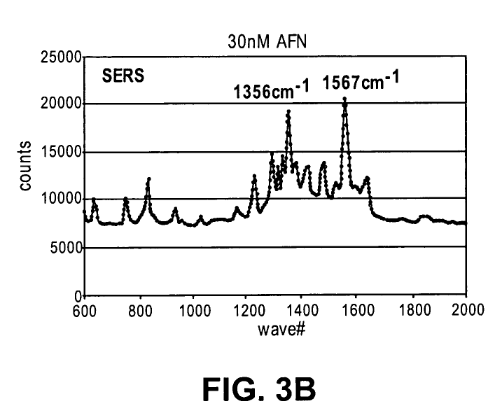

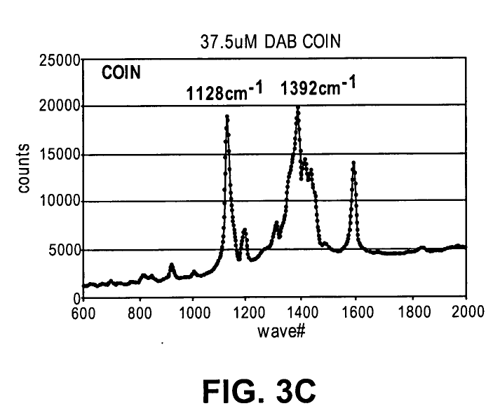

FIGS. 3 A-C provide representative Raman spectra for COINs incorporating several of the organic Raman labels shown in Table 2. Spectra were obtained on a Mattec Renishaw Raman system.

In general, COINs can be prepared by causing colloidal metallic nanoparticles to aggregate in the presence of an organic Raman label. The colloidal metal nanoparticles can vary in size, but are chosen to be smaller than the desired size of the resulting COINs. For some applications, for example, in the oven and reflux synthesis methods, silver particles ranging in average diameter from about 3 to about 12 nm were used to form silver COINs and gold nanoparticles ranging from about 13 to about 15 nm were used to make gold COINs. In another application, for example, silver particles having a broad size distribution of about 10 to about 80 nm were used in a cold synthesis method. Additionally, multi-metal nanoparticles may be used, such as, for example, silver nanoparticles having gold cores.

For organic Raman label compounds that tend to not cause colloid aggregation, an aggregation-inducing agent can be used. For example, the aggregation-inducing agent can be a salt, such as LiCl or NaCl, an acid or a base, such as HNO₃, HCl, or NaOH, or an organic compound, such as adenine, or benzyl-adenine. When aggregation-inducing agents are used, COIN synthesis can be performed at room temperature. Performing synthesis at room temperature is useful for making COINs from fluorescent dyes, since some of them can be unstable at elevated temperatures.

In general, for applications using COINs as reporters for analyte detection, the average diameter of the COIN particle should be less than about 200 nm. Typically, in analyte detection applications, COINs will range in average diameter from about 30 to about 200 nm. More preferably COINs range in average diameter from about 40 to about 200 nm, and more preferably from about 50 to about 200 nm, more preferably from about 50 to about 150 nm, and more preferably about 50 to about 100 nm. The thickness of the coating is, in one aspect, limited by the weight of the resulting particle and its ability to remain suspended in solution. For example, coatings that are lighter, such as protein coatings, can be thicker than heavier silica and metal coatings. Typically, coatings that are less than about 100 nm thick yield COINs that can be suspended in solution. Depending on the application desired, coatings can be as thin as about one layer of molecules.

Typical coatings useful in embodiments of the present invention include coatings such as metal layers, adsorption layers, silica layers, hematite layers, organic layers, and organic thiol-containing layers. Typically, the metal layer is different from the metal used to form the COIN. Additionally, a metal layer can typically be placed underneath any of the other types of layers. Many of the layers, such as the adsorption layers and the organic layers provide additional mechanisms for probe attachment. For instance, layers presenting carboxylic acid functional groups allow the covalent coupling of a biological probe, such as an antibody, through an amine group on the antibody.

To prepare nanoparticles coated with a second metal, COINs are placed in an aqueous solution containing a suitable second metal (as a cation) and a reducing agent. The components of the solution are then subject to conditions that reduce the second metallic cations, thereby forming a metallic layer overlying the surface of the nanoparticle. Metal-coated COINs can be isolated and/or enriched in the same manner as uncoated COINs. In addition, COINs can be coated with a layer of gold by means of epitaxy growth. A procedure for growing gold particles developed by Zsigmondy and Thiessen (Das Kolloide Gold (Leipzig, 1925)), for example, may be employed. The growth medium contains chlorauric acid and hydroxylamine. The thickness of the gold coating can be controlled by the concentration of the COIN particles added to the growth medium.

COINs and metal-coated COINs can be functionalized through attachment of organic molecules to the surface. For example, gold and silver surfaces can be functionalized with a thiol-containing organic molecule to create an organic thiol layer. An organic molecule can be attached through well-known gold-thiol chemistry. The organic molecule can also contain a carboxyl group at the end distal from the thiol enabling further derivatization, such as attachment of a linker molecule, coating, a nucleic acid, or probe. In certain embodiments, the organic thiol-containing molecule is a branched or straight-chain carbon-containing molecule having 2 to about 20 carbon atoms. In additional embodiments, the organic thiol-containing molecule is a polymer, such as for example a polyethylene glycol, a polysaccharide, a peptide containing cysteine, or a mixture thereof. In further embodiments, the organic thiol-containing molecule is capable of binding to a single COIN via two or more thiol groups. In several non-limiting examples, the thiol can be, 2,3-disulfanyl-1-propanol, 3,4-disulfanyl-1-butanol, 4,5-disulfanyl-1-pentanol, 4-amino-2-thiomethyl-butanethiol, or 5-amino-2-thiomethyl-hexanethiol. In general, useful thiol-containing organic molecules have a molecular weight of less than about 9,000. However, in the case of soluble polymers having thiol groups, such as polycysteine, peptides containing cysteine, peptides containing homocysteine, polysaccharides containing thiol groups, or polyethylene glycol polymers containing thiol group(s), the molecular weight can be about 10,000 or less. The organic thiol-containing molecule may also contain one or more additional functional groups, such as groups that allow for coupling with a probe. Useful additional functional groups include, for example, carboxyl groups, esters, amines, photolabile groups, and alcohols. Additionally, suitable functional groups include, but are not limited to, hydrazide, amide, chloromethyl, epoxy, tosyl, and the like, which can be coupled to molecules such as probes through reactions commonly used in the art. Photolabile groups, by which is meant a functional group that can be activated by applying electromagnetic radiation (usually near IR, ultraviolet, or visible light) at a specific wavelength, include, for example, the types of groups disclosed in Aslam, M. and Dent, A., Bioconjugation: Protein Coupling Techniques for the Biomedical Sciences, Grove's Dictionaries, Inc., 301-316 (1998). These functional groups may also be further modified after attachment to a COIN to form more reactive species for coupling, such as for example, oxidizing an alcohol to an aldehyde. Useful thiol-containing molecules include, for example, sulfanylacetic acid, 3-sulfanylpropanoic acid, 6-sulfanylhexanoic acid, 5-sulfanylhexanoic acid, 4-sulfanylhexanoic acid, 3-sulfanylpropylacetate, 3-sulfanyl-1,2-propanediol (1-thioglycerol), 4-sulfanyl-2-butanol, 3-sulfanyl-1-propanol (3-mercapto-1-propanol), ethyl-3-sulfanylpropanoate, cysteine, homocysteine, 2-aminoethanethiol, 4-aminobutanethiol, 4-amino-2-ethyl-butanethiol, and other similar organic molecules having a molecular weight of about 9,000 or less. Additionally, the organic thiol layer may be composed of mixtures of different organic thiol-containing molecules. The mixtures of organic thiol-containing molecules may include thiols having an additional functional group, such as one for probe attachment, and thiols not having an additional functional group, as well as mixtures of thiols containing different functional groups or comprised of different organic molecules. In a further embodiment, the thiol-containing organic layer is attached to a COIN comprised of gold or silver or a COIN having a gold or a silver metal layer. Synthesis of organic thiol layers can be accomplished by standard techniques, such as placing the COINs to be coated in an aqueous solution containing the organic thiol.

Additionally, COINs can be coated with an adsorption layer. The adsorption layer can be comprised of, for example, an organic molecule or a polymer, such as for example, a block co-polymer or a biopolymer, such as for example, a protein, peptide, or a polysaccharide. The adsorption layer, in some cases, stabilizes the COINs and facilitates the reduction or prevention of further aggregation and precipitation from solution. This layer also can provide bio-compatible functional surfaces for probe attachment and aid in the prevention of non-specific binding to the COIN.

The COIN with or without a metal layer can be coated with an adsorbed layer of protein. Suitable proteins include non-enzymatic soluble globular or fibrous proteins. For applications involving molecular detection, the protein should be chosen so that it does not interfere with a detection assay, in other words, the proteins should not also function as competing or interfering probes in a user-defined assay. By non-enzymatic proteins is meant molecules that do not ordinarily function as biological catalysts. Examples of suitable proteins include avidin, streptavidin, bovine serum albumen (BSA), transferrin, insulin, soybean protein, casine, gelatine, and the like, and mixtures thereof. A bovine serum albumen layer affords several potential functional groups, such as, carboxylic acids, amines, and thiols, for further functionalization or probe attachment. Optionally, the protein layer can be cross-linked with EDC, or with glutaraldehyde followed by reduction with sodium borohydride.

As an alternative to metallic protection layers or in addition to metallic protection layers, COINs can be coated with a layer of silica. Silica deposition is initiated from a supersaturated silica solution, followed by growth of a silica layer through ammonia-catalyzed hydrolysis of tetraethyl orthosilicate (TEOS). COINs can be coated with silica and functionalized, for example, with an organic amine-containing group. A silver COIN or silver- or gold-coated COIN can be coated with a layer of silica via the procedure described in V. V. Hardikar and E. Matijevic, J. Colloid Interface Science, 221:133-136 (2000). Additionally, silica-coated COINs are readily functionalized using standard silica chemistry. For example, a silica-coated COIN can be derivatized with (3-aminopropyl)triethoxysilane to yield a silica coated COIN that presents an amine group for further coating, layering, modification, or probe attachment. See, for example, Wong, C., Burgess, J., Ostafin, A., “Modifying the Surface Chemistry of Silica Nano-Shells for Immunoassays,” Journal of Young Investigators, 6:1 (2002), and Ye, Z., Tan, M., Wang, G., Yuan, J., “Preparation, Characterization, and Time-Resolved Fluorometric Application of Silica-Coated Terbium(III) Fluorescent Nanoparticles,” Anal. Chem., 76:513 (2004). Additional layers or coatings that may be layered on a silica coating include the coatings and layers exemplified herein.

COINs can also include an organic layer. This organic layer can overlie another layer, such as a metal layer or a silica layer. An organic layer can also be used to provide colloidal stability and functional groups for further modification. The organic layer is optionally cross-linked to form a more unified coating. An exemplary organic layer is produced by adsorption of an octylamine modified polyacrylic acid onto COINs, the adsorption being facilitated by the positively charged amine groups. The carboxyl groups of the polymer are then cross-linked with a suitable agent such as lysine, (1,6)-diaminoheptane, or the like. Unreacted carboxyl groups can be used for further derivation or probe attachment, such as through EDC coupling.

Further, biomolecules, compounds, or molecules can be attached to COINs through adsorption of the probe to the COIN surface. Alternatively, COINs may be coupled with probes through biotin-avidin coupling. For example, avidin or streptavidin (or an analog thereof) can be adsorbed to the surface of the COIN and a biotin-modified probe contacted with the avidin or streptavidin-modified surface forming a biotin-avidin (or biotin-streptavidin) linkage. As discussed above, optionally, avidin or streptavidin may be adsorbed in combination with another protein, such as BSA, and/or optionally cross-linked. In addition, for COINs having a functional layer that includes a carboxylic acid or amine functional group, probes having a corresponding amine or carboxylic acid functional group can be attached through water-soluble carbodiimide coupling reagents, such as EDC (1-ethyl-3-(3-dimethyl aminopropyl)carbodiimide), which couples carboxylic acid functional groups with amine groups. Further, functional layers and probes can be provided that possess reactive groups such as, esters, hydroxyl, hydrazide, amide, chloromethyl, aldehyde, epoxy, tosyl, thiol, and the like, which can be joined through the use of coupling reactions commonly used in the art. For example, Aslam, M and Dent, A, Bioconjugation: Protein Coupling Techniques for the Biomedical Sciences, Grove's Dictionaries, Inc., (1998) provides additional methods for coupling biomolecules, such as, for example, thiol maleimide coupling reactions, amine carboxylic acid coupling reactions, amine aldehyde coupling reactions, biotin avidin (and derivatives) coupling reactions, and coupling reactions involving amines and photoactivatable heterobifunctional reagents.

Because Raman signal intensity for a particular COIN embodiment varies linearly with concentration, the empirical knowledge of three or more concentration-related intensity values allows the determination of the concentration of an unknown sample of the COIN embodiment.

Specific binding is the specific recognition of one of two different molecules (a specific binding partner) for the other (specific binding partner) and substantially less recognition for other molecules. Generally, the molecules have areas on their surfaces or in cavities giving rise to specific recognition between the two molecules. A ligand is a molecule that binds to another molecule, usually referred to as a receptor. Usually, the term ligand is given to the smaller of the two molecules in the ligand-receptor pair, but it is not necessary for the purposes of the present invention for this to be the case. Exemplary specific binding partners and or ligand-receptor pairs include antibody antigen, enzyme substrate, lectin sugar, hormone or neurotransmitter receptor, and polynucleotide hybridization interactions.

Non-specific binding is non-covalent binding between molecules that is relatively independent of specific surface structures. Non-specific binding may result from several factors including hydrophobic interactions between molecules.

As used herein, the term antibody is used in its broadest sense to include polyclonal and monoclonal antibodies, as well as antigen binding fragments of such antibodies. An antibody (or affinity binding partner) useful the present invention, or an antigen binding fragment thereof, is characterized, for example, by having specific binding activity for an epitope of an analyte. An antibody, for example, includes naturally occurring antibodies as well as non-naturally occurring antibodies, including, for example, single chain antibodies, chimeric, CDR-grafted, bifunctional, and humanized antibodies, as well as antigen-binding fragments thereof. Such non-naturally occurring antibodies can be constructed using solid phase peptide synthesis, can be produced recombinantly, or can be obtained, for example, by screening combinatorial libraries consisting of variable heavy chains and variable light chains.

A marker molecule is a molecule present in a system that allows the detection and or identification of a disease state. Disease markers may be a genetic host factor predisposing to the disease or the occurrence of cell-surface markers, enzymes, or other components, either in altered forms, abnormal concentrations or with abnormal tissue distribution. For example, tumor markers are frequently substances that can be detected in higher-than-normal amounts in blood, urine, or body tissue of some animals with certain types of cancer. The tumor marker may be made by the tumor itself or by the body in response to the tumor. The tumor marker level may also indicate the extent or stage of the disease, how quickly the cancer is likely to progress, and the prognosis.

In general, peptides are polymers of amino acids, amino acid mimics or derivatives, and/or unnatural amino acids. The amino acids can be any amino acids, including α, β, or ω-amino acids and modified amino acids. When the amino acids are α-amino acids, either the L-optical isomer or the D-optical isomer may be used. A peptide can alternatively be referred to as a polypeptide. Peptides contain two or more amino acid monomers, and often more than 50 amino acid monomers (building blocks).

A protein is a long polymer of amino acids linked via peptide bonds and which may be composed of one or more polypeptide chains. More specifically, the term protein refers to a molecule comprised of one or more polymers of amino acids. Proteins are essential for the structure, function, and regulation of the body's cells, tissues, and organs, and each protein has unique functions. Examples of proteins include some hormones, enzymes, and antibodies.

An enzyme is a protein that acts as a catalyst toward a molecule termed the enzyme substrate. An inhibitor is a substance that diminishes the rate of a chemical reaction and an activator is a substance that increases the rate of chemical reaction for a catalyzed chemical reaction. Enzyme activity can be quantitated, for example, through the application of standard kinetics analyses that typically involve the measurement of substrate and or product concentrations over time.

In general, an analyte may be a substance found directly in a sample such as a body fluid from a host. The sample can be examined directly or may be pretreated to render the analyte more readily detectible. Furthermore, the analyte of interest may be determined by detecting an agent probative of the analyte of interest such as a specific binding pair member complementary to the analyte of interest, whose presence will be detected only when the analyte of interest is present in a sample. Thus, the agent probative of the analyte becomes the analyte that is detected in an assay. The body fluid can be, for example, urine, blood, plasma, serum, saliva, semen, stool, sputum, cerebral spinal fluid, tears, mucus, and the like.

An array is an intentionally-created collection of molecules attached to a solid support in which the identity or source of a group of molecules is known based on its location on the array. The molecules housed on the array and within a feature of an array can be identical to or different from each other.

The features, regions, or sectors of an array may have any convenient shape, for example, circular, square, rectangular, elliptical, or wedge-shaped. In some embodiments, the region in which each distinct molecule is synthesized within a sector is smaller than about 1 mm², or less than 0.5 mm². In further embodiments the regions have an area less than about 10,000 μm² or less than 2.5 μm². In general, an array can have any number of features, and the number of features contained in an array may be selected to address such considerations as, for example, experimental objectives, information-gathering objectives, and cost effectiveness. An array could be, for example, a 20×20 matrix having 400 regions, 64×32 matrix having 2,048 regions, or a 640×320 array having 204,800 regions. Advantageously, the present invention is not limited to a particular size or configuration for the array.

FIG. 4 diagrams a method in which enzyme activity in a test sample can be detected and quantified. In this exemplary method diagrammed in FIG. 4, the test sample is a cellular extract and the enzyme activity that is analyzed is protein kinase (or alternately, phosphorylase) activity. Protein kinases are an important class of enzymes involved in cellular signaling and cell growth control. Conjugating biopolymers, such as, fur example, peptides, nucleic acids, and polysaccharides, to COINs allows enzyme substrate specificities and enzyme activities to be examined. In this example, the conjugated peptide having a specific known sequence (a probe peptide) is phosphorylated in the presence of a cellular extract containing a kinase that is capable of phosphorylating the peptide and ATP (adenosine triphosphate). A second COIN or other surface is provided having an attached antibody or affinity binding partner specific for the phosphorylated peptide. Other surfaces include, for example, surfaces suitable for forming an array of specific binding partners, a reporter particle, such as for example a COIN or quantum dot, a microbead, or a magnetic particle. Uncomplexed COINs are removed (by, for example, size separation techniques or in the case of an array, washing the surface of the array) and the conjugated antibody-phosphorylated peptide complex is detected, for example, by Raman spectroscopy. The linearity of the COIN Raman signal intensity with concentration allows the quantitative assessment of enzyme activity within a test sample. Further, the distinctive signatures of the various COINs allow enzymatic assays to be performed in a multiplex fashion. For multiplex analysis, COINs having distinct signatures are provided with different probe peptides (or other biopolymers) so that activity toward a particular biopolymer can be detected by the COIN signature detected. Phosphate-specific antibodies are available commercially, for example, from Cell Signaling Technology, Danvers, Mass. Other enzymatic activities can also be similarly assayed, through selection of a desired substrate probe and corresponding specific binding partner (such as for example, antibodies, nucleic acids, receptors, and lectins) for the modified substrate. In general, enzymatic assay methods are important tools for understanding cell growth regulation (for example, in cancer biology such as through detection of post-translational modification of nucleic acids) and identifying drug targets and screening for drug candidates.

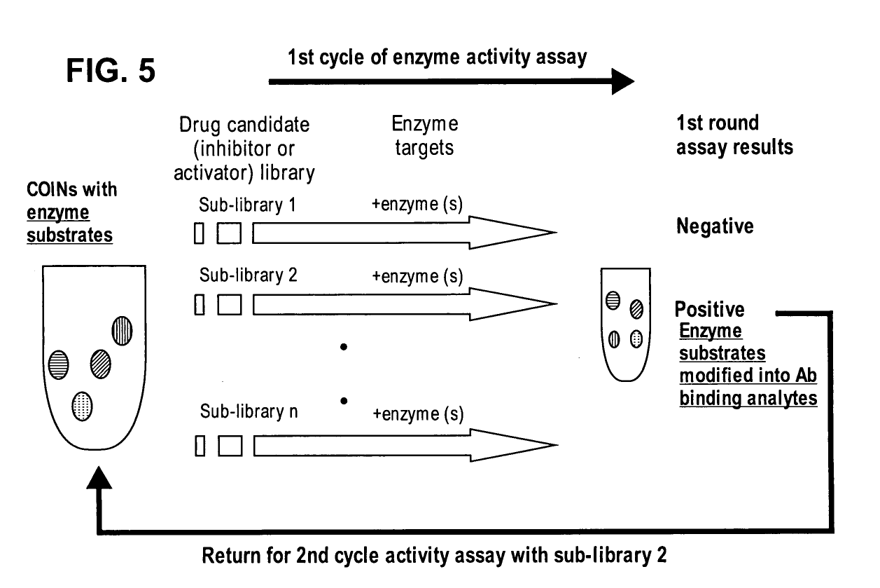

FIG. 5 further elaborates on the capabilities of embodiments of the invention to investigate the activities of compounds or molecules as inhibitors or activators of target enzymes. In this exemplary method, a compound or molecule that is to be tested for its properties as an enzyme inhibitor or activator is added to, for example, an enzyme activity assay as diagrammed in FIG. 4. COINs having attached enzyme substrates are allowed to react with target enzymes in the presence of the molecules or compounds to be tested. Modified COIN-attached enzyme substrates are detected by forming a complex with antibodies specific for the reacted substrate attached to a second COIN or other surface. Other surfaces include, for example, surfaces suitable for forming an array of antibodies or other specific affinity capture molecules, a different type of reporter particle, such as for example a quantum dot, or a magnetic particle. The conjugated antibody-modified substrate complex is detected, for example, by Raman spectroscopy. Libraries of compounds can be tested for potential drug candidates. Drug candidates can be tested in enzymatic reactions to determine their effect on enzymatic activity. Effective candidate compounds are identified according to the correlation of the enzymatic activity changes, as measured, for example using Raman spectroscopy, and the candidate compound concentrations. A library of compounds to be tested can be subdivided into smaller groups. When activity is found in a group that group can then be subdivided and tested until the molecule(s) with activity are identified. This method also allows amount of inhibition or activation to be measured as a function of inhibitor or activator concentration.

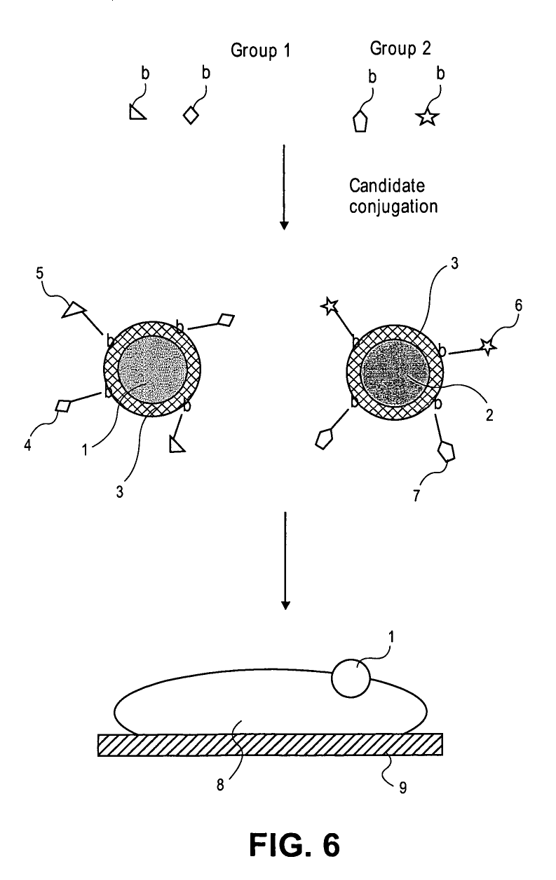

In the embodiment of the invention shown in FIG. 6, COINs are used as carriers for candidate molecules. In this example COINs 1 and 2 are shown as spheres for simplicity, although the metal particle nanoclusters are not necessarily perfectly spherical or even spherical in shape, having a coating layer 3 to which candidate molecules 4, 5, 6, and 7 are conjugated. COINs 1 and 2 have different Raman signatures. The candidate molecules 4, 5, 6, and 7 are attached to the coating layer 3 through a biotin-avidin bond, although other methods of attachment, as described above, can be used. The candidate molecules are compounds that are to be tested for activity on an immobilized cells or tissue sample(s). Candidate molecules could be, for example, antibodies, antigens, inhibitors, activators, cofactors, hormones, peptides, carbohydrates (sugars and polysaccharides), drugs, ligands, antimicrobial compounds, or markers for disease states. Drug candidates can be, for example, ligands for cell surface receptors. In the exemplary method of FIG. 6, a pool of candidate molecules can be tested for activity. To screen a large candidate compound pool for potent drug candidates, the pool can be sub-divided into a manageable number of sub-groups (sub-pools). Different sub-groups of compounds can be attached (covalently or non-covalently) to different types of COINs, wherein one sub-group is attached to one type of COIN. The different COIN types containing the attached sub-groups can then be mixed for binding assay in a single reaction. For example, if there are 100 sub-groups, each containing 100 compounds, there would be 10,000 compounds in a single reaction (requiring 100 COIN types). Further, the binding assay can be performed on microarray containing, for example, immobilized antibodies, receptors, cells, or tissues. If a protein array has 10,000 different protein species, then the system would allow the screening of 10⁴ compounds against 10⁴ potential binding targets. The conjugated COIN types are mixed and the mixture is used to perform a surface binding assay. Unbound COINs are washed from the sample surface and Raman signatures from specifically bound COINs can be detected. Bound COINs indicate that candidate molecules from a group are capable of interacting with, in the case of immobilized cells 8 (FIG. 6), for example, cell surface features. This process can be repeated, dividing the groups further to determine the candidate molecules that are capable of binding to a particular cell surface. If there is a positive interaction, the positive sub-group of candidate compounds needs to be divided into 100 individual compounds for further studies. The immobilized cell can be any type of animal or plant cell, or unicellular organism. For example, an animal cell could be a mammalian cell such as an immune cell, a cancer cell, a cell bearing a blood group antigen such as A, B, D, etc., or an HLA antigen, or virus-infected cell. Further, the target cell could be a microorganism, for example, bacterium, algae, virus, or protozoan. The feature recognized by the probe is present on the surface of the cell and the cell is detected by the presence of a known surface feature (the analyte) through the specific complexation of a COIN to the target cell-surface feature.

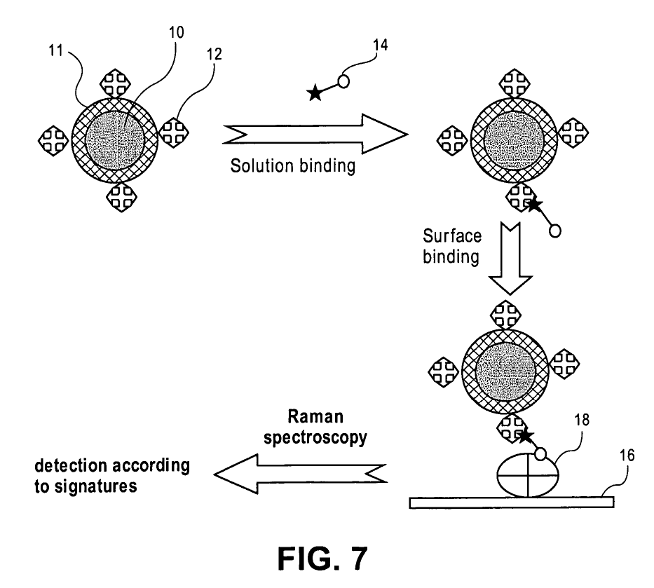

Referring now to FIG. 7, an additional method for screening candidate molecules is provided. In this method, COINs 10 having a coating layer 11 are conjugated to known biomarkers 12, such as for example, receptors, antibodies, and or antigens specific for candidate molecules are allowed to interact with a solution containing candidate molecules. The solution containing candidate molecules 14 could be for example, a serum sample from a patient. Candidate molecules are, for example, antibodies, antigens, drugs designed for human or animal use, metabolites, or markers for disease states in living organisms. In the case of antigen-containing COINs, when there are corresponding antibodies present in the sample, they will bind to COINs through antibody-antigen binding. After removing unbound antibodies from the sample solution (by, for example, centrifugation, chromatography, or magnetic field (in the case of COINs having a magnetic core), the COIN-antigen-antibody complexes can be captured on a surface 16 coated with a capture molecule 18, such as for example, protein A or protein G, antibodies specific for a different epitope of the antigen, or anti-human antibodies). COINs without attached antibodies are removed by washing. The presence of specific antibodies in the samples is detected by the presence of specific COIN signals. This assay can be applied, for example, to diagnosis of auto-immune diseases, cancers, as well as to drug efficacy tests.

Cell surface targets include molecules that are found attached to or protruding from the surface of a cell, such as, proteins, including receptors, antibodies, and glycoproteins, lechtins, antigens, peptides, fatty acids, and carbohydrates. The cellular analyte may be found, for example, directly in a sample such as fluid from a target organism. The sample can be examined directly or may be pretreated to render the analyte more readily detectible. The fluid can be, for example, urine, blood, plasma, serum, saliva, semen, stool, sputum, cerebral spinal fluid, tears, mucus, and the like. The sample could also be, for example, tissue from a target organism.

In the practice of embodiments of the present invention, a Raman spectrometer can be part of a detection unit designed to detect and quantify nanoparticles of the present invention by Raman spectroscopy. Methods for detection of Raman labeled analytes, for example nucleotides, using Raman spectroscopy are known in the art. (See, for example, U.S. Pat. Nos. 5,306,403; 6,002,471; 6,174,677). A non-limiting example of a Raman detection unit is disclosed in U.S. Pat. No. 6,002,471. An excitation beam is generated by either a frequency doubled Nd:YAG laser at 532 nm wavelength or a frequency doubled Ti:sapphire laser at 365 nm wavelength. Pulsed laser beams or continuous laser beams may be used. The excitation beam passes through confocal optics and a microscope objective, and is focused onto the flow path and/or the flow-through cell. The Raman emission light from the labeled nanoparticles is collected by the microscope objective and the confocal optics and is coupled to a monochromator for spectral dissociation. The confocal optics includes a combination of dichroic filters, barrier filters, confocal pinholes, lenses, and mirrors for reducing the background signal. Standard full field optics can be used as well as confocal optics. The Raman emission signal is detected by a Raman detector, which includes an avalanche photodiode interfaced with a computer for counting and digitization of the signal.

Another example of a Raman detection unit is disclosed in U.S. Pat. No. 5,306,403, including a Spex Model 1403 double-grating spectrophotometer with a gallium-arsenide photomultiplier tube (RCA Model C31034 or Burle Industries Model C3103402) operated in the single-photon counting mode. The excitation source includes a 514.5 nm line argon-ion laser from SpectraPhysics, Model 166, and a 647.1 nm line of a krypton-ion laser (Innova 70, Coherent).

Alternate excitation sources include a nitrogen laser (Laser Science Inc.) at 337 nm and a helium-cadmium laser (Liconox) at 325 nm (U.S. Pat. No. 6,174,677), a light emitting diode, an Nd:YLF laser, and/or various ions lasers and/or dye lasers. The excitation beam may be spectrally purified with a bandpass filter (Corion) and may be focused on the flow path and/or flow-through cell using a 6× objective lens (Newport, Model L6X). The objective lens may be used to both excite the Raman-active organic compounds of the COINs and to collect the Raman signal, by using a holographic beam splitter (Kaiser Optical Systems, Inc., Model HB 647-26N18) to produce a right-angle geometry for the excitation beam and the emitted Raman signal. A holographic notch filter (Kaiser Optical Systems, Inc.) may be used to reduce Rayleigh scattered radiation. Alternative Raman detectors include an ISA HR-320 spectrograph equipped with a red-enhanced intensified charge-coupled device (RE-ICCD) detection system (Princeton Instruments). Other types of detectors may be used, such as Fourier-transform spectrographs (based on Michaelson interferometers), charged injection devices, photodiode arrays, InGaAs detectors, electron-multiplied CCD, intensified CCD and/or phototransistor arrays.

Any suitable form or configuration of Raman spectroscopy or related techniques known in the art may be used for detection of the nanoparticles of the present invention, including but not limited to normal Raman scattering, resonance Raman scattering, surface enhanced Raman scattering, surface enhanced resonance Raman scattering, coherent anti-Stokes Raman spectroscopy (CARS), stimulated Raman scattering, inverse Raman spectroscopy, stimulated gain Raman spectroscopy, hyper-Raman scattering, molecular optical laser examiner (MOLE) or Raman microprobe or Raman microscopy or confocal Raman microspectrometry, three-dimensional or scanning Raman, Raman saturation spectroscopy, time resolved resonance Raman, Raman decoupling spectroscopy or UV-Raman microscopy.

Raman signatures from COINs can be analyzed, for example, using data signature and peak analysis through peak fitting. Scanned data were analyzed for signature profiles such as Raman peak intensities and locations as well as peak width. The data were analyzed using peak and curve fitting algorithms to identify statistically the most likely parameters (such as for example, wave number, intensity, peak width, and associated baseline values) that come from control experiments, such as for example, signals from water, solvent, the substrate, and or system noise. Raman peak intensities were normalized to methanol's first main peak and, where required, also further normalized to label concentrations.

### EXAMPLE 1

Synthesis

Chemical reagents: Biological reagents including anti-IL-2 and anti-IL-8 antibodies were purchased from BD Biosciences Inc. The capture antibodies were monoclonal antibodies generated from mouse. Detection antibodies were polyclonal antibodies generated from mouse and conjugated with biotin. Aqueous salt solutions and buffers were purchased from Ambion, Inc. (Austin, Tex., USA), including 5 M NaCl, 10×PBS (1×PBS 137 mM NaCl, 2.7 mM KCl, 8 mM Na₂HPO₄, and 2 mM KH₂PO₄, pH 7.4). Unless otherwise indicated, all other chemicals were purchased, at highest available quality, from Sigma Aldrich Chemical Co. (St. Louis, Mo., USA). Deionized water used for experiments had a resistance of 18.2×10⁶ Ohms-cm and was obtained with a water purification unit (Nanopure Infinity, Barnstad, USA).

Silver seed particle synthesis: Stock solutions (0.500 M) of silver nitrate (AgNO₃) and sodium citrate (Na₃Citrate) were filtered twice through 0.2 micron polyamide membrane filters (Schleicher and Schuell, N.H., USA) which were thoroughly rinsed before use. Sodium borohydrate solution (50 mM) was made fresh and used within 2 hours. Silver seed particles were prepared by rapid addition of 50 mL of Solution A (containing 8.00 mM Na₃Citrate, 0.60 mM sodium borohydrate, and 2.00 mM sodium hydroxide) into 50 mL of Solution B (containing 4.00 mM silver nitrate) under vigorous stirring. Addition of Solution B into Solution A led to a more polydispersed suspension. Silver seed suspensions were stored in the dark and used within one week. Before use, the suspension was analyzed by Photon Correlation Spectroscopy (PCS, Zetasizer 3000 HS or Nano-ZS, Malvern) to ensure the intensity-averaged diameter (z-average) was between 10-12 nm with a polydispersity index of less than 0.25.

Gold seed particle synthesis: A household microwave oven (1350W, Panasonic) was used to prepare gold nanoparticles. Typically, 40 mL of an aqueous solution containing 0.500 mM HAuCl₄ and 2.0 mM sodium citrate in a glass bottle (100 mL) was heated to boiling in the microwave using the maximum power, followed by a lower power setting to keep the solution gently boiling for 5 min. 2.0 grams of PTFE boiling stones (6 mm, Saint-Gobain A1069103, through VWR) were added to the solution to promote gentle and efficient boiling. The resultant solutions had a rosy red color. Measurements by PCS showed that the gold solutions had a typical z-average of 13 nm with a polydispersity index of less than 0.04.

COIN Synthesis: In general, Raman labels were pipetted into the COIN synthesis solution to yield final concentrations of the labels in synthesis solution of about 1 to about 50 μM. Stock solutions of Raman labels were prepared having concentrations of about 0.25 mM to about 1 mM of ultra-purified water. In some cases, acid or organic solvents, such as, for example, ethanol, were used to enhance label solubility. For example, 8-aza-adenine and N-benzoyladenine were pipetted into the COIN formation reaction as 1.00 mM solutions in 1 mM HCl, 2-mercapto-benzimidazole was added from a 1.0 mM solution in ethanol, and 4-amino-pyrazolo[3,4-d]pyrimidine and zeatin were added from a 0.25 mM solution in 1 mM HNO₃.

Reflux method: To prepare COIN particles with silver seeds, typically, 50 mL silver seed suspension (equivalent to 2.0 mM Ag⁺) was heated to boiling in a reflux system before introducing Raman labels. Silver nitrate stock solution (0.50 M) was then added dropwise or in small aliquots (50-100 μL) to induce the growth and aggregation of silver seed particles. Up to a total of 2.5 mM silver nitrate could be added. The solution was kept boiling until the suspension became very turbid and dark brown in color. At this point, the temperature was lowered quickly by transferring the colloid solution into a glass bottle. The solution was then stored at room temperature. The optimum heating time depended on the nature of Raman labels and amounts of silver nitrate added. It was found helpful to verify that particles had reached a desired size range (80-100 nm on average) by PCS or UV-Vis spectroscopy before the heating was arrested. Normally, a dark brown color was an indication of cluster formation and associated Raman activity.

To prepare COIN particles with gold seeds, typically, gold seeds were first prepared from 0.25 mM HAuCl₄ in the presence of a Raman label (for example, 20 μM 8-aza-adenine). After heating the gold seed solution to boiling, silver nitrate and sodium citrate stock solutions (0.50 M) were added, separately, so that the final gold suspension contained 1.0 mM AgNO₃ and 1.0 mM sodium citrate. Silver chloride precipitate might form immediately after silver nitrate addition but disappeared soon with heating. After boiling, an orange-brown color developed and stabilized. An additional aliquot (50-100 μL) of silver nitrate and sodium citrate stock solutions (0.50 M each) was added to induce the development of a green color, which was the indication of cluster formation and was associated with Raman activity.

Note that the two procedures produced COINs with different colors, primarily due to differences in the size of primary particles before cluster formation.

Oven method: COINs can also be prepared conveniently by using a convection oven. Silver seed suspension was mixed with sodium citrate and silver nitrate solutions in a 20 mL glass vial. The final volume of the mixture was typically 10 mL, which contained silver particles (equivalent to 0.5 mM Ag⁺), 1.0 mM silver nitrate and 2.0 mM sodium citrate (including the portion from the seed suspension). The glass vials were incubated in the oven, set at 95° C., for 60 min. before being stored at room temperature. A range of label concentrations could be tested at the same time. Batches showing brownish color with turbidity were tested for Raman activity and colloidal stability. Batches with significant sedimentation (which occurred when the label concentrations were too high) were discarded. Occasionally, batches that did not show sufficient turbidity could be kept at room temperature for an extended period of time (up to 3 days) to allow cluster formation. In many cases, suspensions became more turbid over time due to aggregation, and strong Raman activity developed within 24 hours. A stabilizing agent, such as bovine serum albumin (BSA), could be used to stop the aggregation and stabilize the COIN suspension.

A similar approach was used to prepare COINs with gold cores. Briefly, 3 mL of gold suspensions (0.50 mM Au³⁺) prepared in the presence of Raman labels was mixed with 7 mL of silver citrate solution (containing 5.0 mM silver nitrate and 5.0 mM sodium citrate before mixing) in a 20 mL glass vial. The vial was placed in a convection oven and heated to 95° C. for 1 hour. Different concentrations of labeled gold seeds could be used simultaneously in order to produce batches with sufficient Raman activities.

Cold Method: 100 mL of silver particles (1 mM silver atoms) were mixed with 1 mL of Raman label solution (typically 1 mM). Then, 5 to 10 mL of 0.5 M LiCl solution was added to induce silver aggregation. As soon as the suspension became visibly darker (due to aggregation), 0.5% BSA was added to inhibit the aggregation process. Afterwards, the suspension was centrifuged at 4500 g for 15 minutes. After removing the supernatant (mostly single particles), the pellet was resuspended in 1 mM sodium citrate solution. The washing procedure was repeated for a total of three times. After the last washing, the resuspended pellets were filtered through 0.2 μM membrane filter to remove large aggregates. The filtrate was collected as COIN suspension. The concentrations of COINs were adjusted to 1.0 or 1.5 mM with 1 mM sodium citrate by comparing the absorbance at 400 nm with 1 mM silver colloids for SERS.

It should be noted that a COIN sample can be heterogeneous in terms of size and Raman activity. We typically used centrifugation (200-2,000×g for 5-10 min.) or filtration (300 kDa, 1000 kDa, or 0.2 micron filters, Pall Life Sciences through VWR) to enrich for particles in the range of 50-100 nm. It is recommended to coat the COIN particles with a protection agent (for example, BSA, antibody) before enrichment. Some lots of COINs that we prepared (with no further treatment after synthesis) were stable for more than 3 months at room temperature without noticeable changes in physical and chemical properties.

Particle size measurement: The sizes of silver and gold seed particles as well as COINs were determined by using Photon Correlation Spectroscopy (PCS, Zetasizer3 3000 HS or Nano-ZS, Malvern). All measurements were conducted at 25° C. using a He—Ne laser at 633 nm. Samples were diluted with DI water when necessary. Some of the COIN samples (with a total silver concentration of 1.5 mM) were diluted ten times with 1 mM sodium citrate before measurement. FIGS. 10A and B show, respectively, the zeta potential measurements of silver particles of initial z-average size of 47 nm (0.10 M) with a suspending medium of 1.00 mM sodium citrate and the evolution of aggregate size (z-average) in the presence of 20 μM 8-aza-adenine.

Raman spectral analysis: for all SERS and COIN assays in solution, a Raman microscope (Renishaw, UK) equipped with a 514 nm Argon ion laser (25 mW) was used. Typically, a drop (50-200 μL) of a sample was placed on an aluminum surface. The laser beam was focused on the top surface of the sample meniscus and photons were collected for about 10-20 seconds. The Raman system normally generated about 600 counts from methanol at 1040 cm⁻¹ for a 10 second collection time. For Raman spectroscopy detection of an analyte immobilized on a surface, Raman spectra were recorded using a Raman microscope built in-house. This Raman microscope consisted of a water cooled Argon ion laser operating in continuous-wave mode, a dichroic reflector, a holographic notch filter, a Czerny-Turner spectrometer, and a liquid nitrogen cooled CCD (charge-coupled device) camera. The spectroscopy components were coupled with a microscope so that the microscope objective focused the laser beam onto a sample, and collected the back-scattered Raman emission. The laser power at the sample was about 60 mW. All Raman spectra were collected with 514 nm excitation wavelength.

Antibody Coating: A 500 μL solution containing 2 ng of a biotinylated anti-human antibody (anti-IL-2 or anti-IL-8) in 1 mM sodium citrate (pH 9) was mixed with 500 μL of a COIN solution (made with 8-aza-adenine or N-benzoyl-adenine); the resulting solution was incubated at room temperature for 1 hour, followed by adding 100 μL of PEG-400 (polyethyleneglycol-400). The solution was incubated at room temperature for another 30 min., then 200 μL of 1% Tween™-20 was added to the solution. The solution was centrifuged at 2000×g for 10 min. After removing the supernatant, the pellet was resuspended in 1 mL solution (BSAT) containing 0.5% BSA, 0.1% Tween-20 and 1 mM sodium citrate. The solution was then centrifuged at 1000×g for 10 min. The BSAT washing procedure was repeated for a total of 3 times. The final pellet was resuspended in 700 μL of diluting solution (0.5% BSA, 1×PBS, 0.05% Tween™-20). The Raman activity of the COINs was measured and adjusted to a specific activity of about 500 photon counts per μl per 10 seconds using a Raman spectroscope that generated about 600 counts from methanol at 1040 cm⁻¹ for 10 second collection time.

What is claimed is:

1. A method for detecting enzyme activity in a sample comprising:
contacting a sample solution to be tested for enzyme activity with a nanocluster of metal particles having a unique Raman signature, wherein the unique Raman signature is produced by at least one Raman active organic compound incorporated within the nanocluster, and having an attached biopolymer, under conditions that allow the biopolymer attached to the nanocluster to be modified by the enzyme to be tested for in the sample;
contacting the biopolymer attached to the nanocluster with a specific binding partner specific for a modified state of the biopolymer, wherein the specific binding partner is attached to a solid surface, under conditions that allow the specific binding partner to specifically attach to the modified state of the biopolymer;
removing nanoclusters that remain uncomplexed to the specific binding partner from any nanoclusters that are complexed to the specific binding partner; and
making at least one Raman measurement in order to detect the presence of a nanocluster of metal particles having a unique Raman signature.

2. The method of claim 1 wherein the sample solution is tested for two types of enzyme activity simultaneously using two types of nanoclusters of metal particles having different unique Raman signatures produced by different Raman active organic compounds incorporated within the nanocluster.

3. The method of claim 1 wherein an amount of enzyme activity in the sample is measured.

4. The method of claim 1 wherein an amount of enzyme activity in the sample is measured for two different enzymes simultaneously.

5. The method of claim 1 wherein the biopolymer is selected from the group consisting of peptides, polysaccharides, and nucleic acids.

6. The method of claim 1 wherein the specific binding partner is selected from the group consisting of antibodies, nucleic acids, receptors, and lectins.

7. The method of claim 1 wherein the enzyme activity tested for is kinase activity, the modified state of the biopolymer is a phosphorylated peptide, and the specific binding partner is an antibody.

8. The method of claim 1 wherein the solid surface is a microsphere, a nanoparticle, or a magnetic particle.

9. The method of claim 1 wherein the solid surface contains an array of antibodies that are specific for more than one type of modified specific binding partner.

10. The method of claim 1 wherein the nanocluster of metal particles is comprised of silver or gold.

11. A method for determining enzymatic activity comprising:
contacting a solution containing an enzyme and a compound to be tested for its effect on enzyme activity with a nanocluster of metal particles having a unique Raman signature, wherein the unique Raman signature is produced by at least one Raman active organic compound incorporated within the nanocluster, and having an attached enzyme substrate, under conditions that allow the enzyme substrate attached to the nanocluster to be modified by the enzyme in the solution;
contacting the nanocluster-attached enzyme substrate with an antibody specific for an enzyme-modified state of the enzyme substrate, wherein the antibody is attached to a solid surface, under conditions that allow the antibody to specifically attach to the modified state of the enzyme substrate;
removing nanoclusters that remain uncomplexed to the antibody from any nanoclusters that are complexed to the antibody; and
making at least one Raman measurement in order to detect the presence of a nanocluster of metal particles having a unique Raman signature.

12. The method of claim 11 wherein the sample solution is tested for two types of enzyme activity simultaneously using two types of nanoclusters of metal particles having different unique Raman signatures produced by different Raman active organic compounds incorporated within the nanocluster.

13. The method of claim 11 wherein an amount of enzyme activity in the sample is measured for two different enzymes simultaneously.

14. The method of claim 11 wherein the solid surface is a microsphere, a nanoparticle, or a magnetic particle.

15. The method of claim 11 wherein the solid surface contains an array of antibodies that are specific for more than one type of modified substrate.

16. The method of claim 11 wherein the nanocluster of metal particles is comprised of silver or gold.

17. The method of claim 11 wherein the solution containing the enzyme additionally contains at least one compound to be tested for its ability to inhibit or activate enzyme activity.

18. The method of claim 11 wherein the solution containing the enzyme additionally contains at least one compound to be tested for its ability to inhibit or activate enzyme activity and an amount of enzyme activity is measured.

19. A method for determining a biologic activity of a plurality of molecules comprising:
attaching a plurality of molecules to be tested for activity to a nanocluster of metal particles having a unique Raman signature, wherein the unique Raman signature is produced by at least one Raman active organic compound incorporated within the nanocluster, and attaching a second plurality of molecules to be tested for activity to a second nanocluster of metal particles having a unique Raman signature different from the Raman signature of the first nanocluster;
contacting a solution of the first and second nanoparticles with an array of cells under conditions that allow the molecules to be tested for activity to interact specifically with surface features of the cells of the array;
removing uncomplexed nanoparticles; and
detecting signatures of nanoclusters complexed to cells of the array using Raman spectroscopy.

20. The method of claim 19 wherein the cells of the array are comprised of immobilized animal tissues.

21. The method of claim 19 wherein regions of the array are comprised of homogeneous cell populations.

22. The method of claim 21 wherein the homogeneous cell populations contain cells derived from animal tissue.

23. The method of claim 19 wherein the molecules to be tested for activity are compounds designed for human or animal use, metabolites, or markers for disease states in living organisms.

24. The method of claim 19 wherein the first and second nanoclusters of metal particles are comprised of silver or gold.

25. A method for determining biologic activity for a sample comprising:
contacting the sample with a nanocluster of metal particles having a unique Raman signature produced by at least one Raman active organic compound incorporated within the nanocluster, and having an attached molecule specific for a candidate molecule, under conditions that allow the attached molecule to selectively bind to a candidate molecule;
separating the nanoclusters from the sample;
contacting the nanoclusters of metal particles with a surface having a second molecule specific for a candidate molecule attached to the surface, under conditions that allow the attached molecule to selectively bind to a candidate molecule;
removing nanoclusters that are not attached to the surface; and
detecting signals of nanoclusters attached to the surface using Raman spectroscopy.

26. The method of claim 25 wherein the plurality of candidate molecules is contacted with a plurality of nanoclusters of metal particles having different unique Raman signatures produced by different Raman active organic compounds incorporated within the nanocluster.

27. The method of claim 25 wherein the plurality of candidate molecules are selected from the group consisting of antibodies, antigens, drugs, metabolites, neurotransmitters, markers for disease states, nucleic acids, and combinations thereof.

28. The method of claim 25 wherein the surface contains an array of regions containing molecules specific for a candidate molecule.

29. The method of claim 25 wherein the plurality of candidate molecules are markers for disease states.

30. The method of claim 25 wherein the first and second nanoclusters of metal particles are comprised of silver or gold.
